(19) United States
(12) Patent Application Publication
(10) Pub. No.: US 2004/0115792 A1
(43) Pub. Date: Jun. 17, 2004

(54) Yeast strain for testing the geno- and cytotoxicity of complex environmental contaminations

(76) Inventor: Hella Lichtenberg-Frate, Bonn (DE)

Correspondence Address: JENKINS & WILSON, PA, 3100 TOWER BLVD, SUITE 1400, DURHAM, NC, 27707, US

(21) Appl. No.: 10/433,640

(22) PCT Filed: Dec. 12, 2001
(86) PCT No.: PCT/EP01/14610

(30) Foreign Application Priority Data
Dec. 12, 2000 (DE) 100 61 872.3

(51) Int. Cl. C12N 1/18

(52) U.S. Cl. 435/254.21

(57) ABSTRACT

The invention relates to modified yeast strains and to methods for constructing yeast strains, as well as to their use for testing the geno- and/or cytotoxicity of complex environmental contamination.

The invention relates to modified yeast strains and to methods for the construction of such yeast strains and to the use thereof for testing the genotoxicity and/or cytotoxicity of complex environmental contaminations. In detail, yeast strains, especially Saccharomyces cerevisiae strains, are disclosed in which the nucleic acid sequences for receptor and reporter signal potencies for genotoxic and cytotoxic environmental contaminations, such as the “green fluorescent” gene from Aequoria victoria, and the “red fluorescent” gene from the Indo-Pacific sea anemone species Discosoma, are stably integrated in the yeast genome. These yeast strains can be employed as the biological component of a biosensor suitable for the dose-dependent genotoxic and cytotoxic substance testing for various, especially organotin, environmental poisons, i.e., the detection of all pollutants occurring in the measuring sample including any toxic degradation products.

### BACKGROUND OF THE INVENTION

Scientific examinations on lower animals yielded an extreme disturbance of the hormonal and morphological reproduction systems from the influence of organotin compounds, which has resulted in the infertility and even extinction of certain species. Since the synthesis of sexual hormones proceeds according to the same principles in the entire animal kingdom and also in humans, a negative effect on then hormonal reproduction system of humans cannot be excluded. In mammals, TBT (tributyltin) influences the hormonal equilibrium through effects on endocrine glands, such as the hypophysis, thyroid gland and the hormone glands of the gonads. The cytochrome-P450-dependent aromatase system plays an important role in the conversion of male sexual hormones (androgens), which are always the precursors of the female sexual hormones (estrogens) in the female sex. In various research studies, it has been demonstrated that TBT interferes with the endogenous steroid metabolism of marine gastropods on the level of cytochrome-P450-dependent aromatase and inhibits the aromatization of androgens into estrogens, as described in R. Bettin et al., Phys. Rev. Stat. Phys. Plasmas Fluids Relat. Interdiscip. Topics 51: 212-219 (1995).

The same enzyme system, which is also referred to as multifunctional oxygenase system (MFO system) and was detected in mollusks as well as in mammals and humans, catalyzes both the aromatization into estrogens and the degradation of TBT. In female animals, the increased androgen content resulting from the inhibition, probably competitive inhibition, of cytochrome-P450-dependent aromatase induces the additional development of male secondary sex characteristics. Due to these results and the fact that the steroid biosynthesis proceeds according to the same principles in the entire animal kingdom, a negative effect of TBT and other organotin compounds on the cytochrome-P450-dependent aromatase system of higher developed organisms cannot be excluded. Possibly, the environmental loading with TBT and other organotin compounds can be considered a factor which is responsible for the continuously increasing reproduction disorders in the female sex both in humans and in animals living in marine or limnic-aquatic habitats. In a study published by the World Wildlife Foundation, USA, reproduction disorders are described in both male and female sexes in over 26 animal species from aquatic biotopes. There are no systematic quantitative and qualitative studies on the accumulation and toxicity of organotin compounds in humans. However, alarming results have been obtained from in vitro studies made by the work group endocrinology of the Institut für Klinische Biochemie of Bonn, Germany, according to which the enzyme aromatase from uterus endothelial cells, which is necessary for the conversion of androgens into estrogens, is completely inhibited already by nanogram amounts (D. Klingmuller, Institut für Klinische Biochemie, Universität Bonn, personal communication).

Severe effects from environmentally relevant chemicals on the hormone balance and fertility in the male and female sexes have been described, for example, for DDE (2,2-bis(4-chlorophenyl)-1,1-dichloroethane; 4,4′-DDE), a degradation product of the agriculturally employed pesticide keltane, manifested by the development of hermaphrodites in male alligators in a lake area in Florida, USA.

In England, the induction of sterility in the male sex by nonylphenols has been detected in fish. Nonylphenols, whose annual production is 20,000 tons in Great Britain alone, are employed in the production of plastics and arrive at first in the waste water and thereafter in surface water bodies, such as creeks, rivers and lakes, in the production and use of plastic materials. The intoxination of humans with TMT (trimethyltin), a potent neurotoxic organotin compound, resulted in epilepsy, amnesia and hippocampal damage, as described in R. G. Feldmann et al., Arch. Neurol. 50: 1320-1324 (1993); S. Kreyberg et al., Clin. Neuropathol. 11: 256-259 (1992).

For TMT and the related TBT, neurotoxic effects selectively affecting hippocampus regions have also been shown, as described in T. J. Walsh et al., Neurobehav. Toxicol. Teratol 4: 177-183 (1982); C. D. Balaban et al., Neuroscience, 28: 337-361 (1988), and K. Tsunashima et al., Synapse 29: 333-342 (1998).

In the opinion of the World Health Organization (WHO), the biocide tributyltin (TBT) belongs to the most toxic substances which have ever been produced and released into the environment. The toxicity and effectiveness of TBT is comparable only to that of dioxin. TBT is mostly produced as an oxide, fluoride, sulfide, chloride or acetate and will ionize in aqueous solutions to form a hydratizing cation which is characterized by a high bioavailability.

Since the beginning of the 1950s, after the discovery of their biocidal effect, organotin compounds have been employed in the industry for the impregnation, stabilization and preservation of a wide variety of products. The main fields of application for TBT and triphenyltin (TPhT) are the use in conventional antifouling paints with contact leaching, in ablative antifouling paints and in self-polishing copolymers. Organotin compounds are employed in large amounts as thermal and/or ultraviolet stabilizers in almost all PVC processing methods (calendering and extrusion methods, blow-molding and injection molding methods). This field of application is the most important by far in terms of quantities, based on all fields of application of organotin compounds. Worldwide, about 75,000 tons/year of organotin stabilizers are employed; in Europe, the consumption is about 15,000 tons/year, and in Germany, it is about 5,000 tons/year (all consumption figures for the year 1999). Due to their biocidal effect, these chemicals are also employed as wood and material protective agents for textiles, sealing and casting compositions (e.g., polyurethane foams), for paints and adhesives as well as mineral materials (e.g., insulating materials) and as plastic stabilizers. Organotin compounds are employed in agriculture, horticulture and animal keeping as biocides against fungi, bacteria, ants, mites, nematodes, insects, mollusks and rodents. In addition, their application in paper and brewing business, on cooling towers, in leather impregnation, in dispersion dyes and as a disinfectant plays a role.

Due to the numerous applications of organotin compounds, humans come into contact with these compounds in different ways. Leaching effects cause:

i) an enormous load on both marine water bodies close to the shore and limnic bodies of water and sediments and thus load on marine foods (food-based oral uptake), as described in R. Nilsson, Toxicol. Pathol. 28: 420-31 (2000);

ii) charging into sweet and drainage waters and a continuous load on waste waters.

Emissions of TBT from impregnated woods and the use as biocides for the protection of textiles, wallpapers, wall paint, paper, mineral insulating materials, silicone and polyurethane foams lead to loads on the indoor air (inhalational uptake through respiration). A possible direct contact for uptake through the skin (percutaneously) is provided by textiles which are impregnated with TBT for protecting the tissue.

In addition to chemical full analysis, supervision of waste water introduction for ecotoxic loadings is effected by standardized biotests (DIN, ISO) with bacteria, algae, planktonic crustaceans and fishes as indicator organisms. To date, organotin compounds have exclusively been detected qualitatively with methods of gas or liquid chromatography, as described in S. Chiron et al., J. Chromatogr. A. 879: 137-45 (2000); E. Gonzalez-Toledo et al., J. Chromatogr. A. 878: 69-76 (2000); E. Millan, Pawliszyn, J. Chromatogr A. 873: 63-71 (2000).

These methods are technically complicated and expensive, the only German supplier of the corresponding analytical systems being the company Galab in Geesthacht. On the ACHEMA fair (July 2000, Frankfurt), the ICB Institut (Münster) in cooperation with the company Gerstel presented the prototype of a gas chromatograph which can detect organotin compounds; one device will probably cost 100,000 DM and be commercially available from about spring 2001. The sensitivity of modern chemical-analytical methods for general environmental toxins including biochemical methods (enzyme assays and immunoassays) is far below the threshold of ecotoxic effects.

Bioassays for the analysis of complex genotoxic and cytotoxic noxious substances including organotin compounds have not been employed to date.

For the detection of general environmental genotoxins, numerous prokaryotic test systems are employed, inter alia. Examples include the Ames test (B. N. Ames et al., Proc. Natl. Acad. Sci. USA 70, 2281-2285 (1973)) and the bacterial SOS-lux test (L. R. Ptitsyn et al., Applied and Environmental Microbiology 63: 4377-4384 (1997) and G. Horneck et al., Biosensors for Environmental Diagnostics, Teubner, Stuttgart, pp. 215-232 (1998)). In the prokaryotic lux-fluoro test, recombinant Salmonella typhimurium TA1535 bacterial cells are employed (P. Rettberg et al., Analytica Chimica Acta 387: 289-296 (1999)).

The existing prokaryotic biotests for the detection of environmental toxins have the following drawbacks:

(i) time-delayed reactions are measured with the dying of the organisms/cells;

(ii) all those substances which induce mutations in higher organisms through their interactions with cell structures (for example, through disturbances of the spindle apparatus) are not covered;

(iii) no genotypically stable differentiated receptor and reporter components for genotoxic and/or cytotoxic agents are contained; and

(iv) active bacterial efflux systems for hydrophobic substances are not switched off (see also H. W. van Ween & W. N. Konings, Biol. Chem. 378: 769-777 (1997), and A. Cloeckert & S. Schwarz, Vet. Res. 32: 301-310 (2001), and M. Daly & S. Fanning, Appl. Environ. Microbiol. 66: 4842-4848 (2000)).

A recently published biotest for the determination of aquatic toxicity is based on the fermenting performance of Saccharomyces cerevisiae cells (J. Weber et al., Z. Umweltchem. Ökotox. 12: 185-189 (2000), and Weber et al., Vom Wasser 95: 97-106 (2000)). However, this process cannot be automated and takes about 25 hours.

In addition, wild type cells of the yeast Saccharomyces cerevisiae express a considerable endogenous resistance against organotin compounds and a wide variety of hydrophobic as well as metal-containing substances, mediated by the ABC (ATP-binding cassette) transporter genes PDR5, SNQ2 and YOR1 (J. Golin et al., Antimicrob. Agents Chemother. 44: 134-138 (2000)). Thus, Saccharomyces cerevisiae wild type strains are not suitable for environmental-biotechnological purposes (the detection of noxious substances relevant to the environment). The advantages of the use of the wild type strains are, in particular:

(A) no defined genetic background;

(B) metabolic measuring parameters are subject to a complex metabolic regulation and thus indicate possible intoxications only inaccurately, since

(C) the expression of the ABC transporters, which are responsible for the endogenous resistance, protects the cells and thus metabolic functions; and

(D) the evaluation of the data obtained in terms of toxicological effects of aqueous solutions and substances is thus rendered difficult.

The intensive use of organotin compounds in a number of industrial processes with increasing release and accumulation in the environment is presently causing considerable ecopolitical and public concern.

On the other hand, however, there is a need in the industry for using these or similar substances in the production process.

Thus, there is an urgent need for a detection method for the quick detection of noxious substances generally relevant to the environment, such as organotin compounds, ionizing and non-ionizing (ultraviolet) radiations, but also chemical carcinogens. The high homology of essential cellular processes in yeast and cells of higher eukaryotes allows to conclude that yeast could be suitable as a eukaryotic detection and analytic system for the identification of genotoxic and cytotoxic compounds which are generally noxious. Such yeast strains could be employed in serial tests for the screening of possibly contaminated solutions on a very small scale with high efficiency (bioassay).

### SUMMARY OF THE INVENTION

It was the object of the present invention to avoid the above mentioned drawbacks and to develop a test system based on yeast cells for the dose-dependent genotoxic and cytotoxic substance testing of noxious substances relevant to the environment and especially of organotin environmental poisons. It has now been found that a genetically defined (isogenic) yeast host strain, e.g., a Saccharomyces cerevisiae yeast host strain, in which genotoxic and cytotoxic signal potencies are stably integrated and expressed in the yeast genome is a suitable test system. In addition, xenobiotic translocation genes which code for ABC transporter genes responsible for the endogenous resistance can be specifically deleted in the yeast host strain. This test system enables the detection of all noxious substances occurring in the measuring sample including possible toxic degradation products.

Thus, the present invention relates to

(1) a modified yeast strain in which

(a) a genotox cassette comprising a first promoter and a first reporter gene functionally linked to the first promoter; and

(b) a cytotox cassette comprising a second promoter and a second reporter gene functionally linked to the second promoter;

wherein the promoters and reporter genes in (1) and (2) are respectively distinct from each other;

are stably and functionally integrated in the genome of a yeast host strain;

(2) a preferred embodiment of (1) wherein the yeast strain has been sensitized by disrupting or deleting one or more of the xenobiotic translocation genes present in the yeast host strain;

(3) a method for the preparation of a modified yeast strain as defined above in (1) or (2), comprising the integration of genotox and cytotox cassettes into the yeast host strain;

(4) a method for the detection of noxious substances relevant to the environment, comprising:

(a) the treatment of a modified yeast strain as defined above in (1) or (2) with a test substance or a mixture of test substances;

(b) determinations of growth in the presence or after completion of the treatment with said test substance/mixture of test substances; and

(c) measurements of the increase or decrease of the reporter gene activity of the yeast strain in the presence or after completion of the treatment with said test substance/mixture of test substances;

(5) the use of a modified yeast strain as defined above in (1) or (2) for testing the genotoxicity and/or cytotoxicity of complex environmental contaminations; and

(6) a test kit and biosensor for testing the genotoxicity and/or cytotoxicity of complex environmental contaminations, comprising a modified yeast strain as defined above in (1) or (2).

The modified yeast host strains according to the invention are suitable, in particular, for the dose-dependent genotoxicity and cytotoxicity testing of substances for various environmental poisons, especially organotin environmental poisons, i.e., the detection of all noxious substances occurring in the measured sample including any toxic degradation products. These yeast cells having receptor and reporter signal potencies stably integrated into the genome can thus be employed in test kits and biosensors for genotoxic and/or cytotoxic environmental contaminations. The cell structure (plasma membrane, intracellular membrane systems, cell organelles, enzyme apparatus) of the yeast is similar, in principle, to the cells of higher organisms. However, due to their easier culturing (doubling time about 90 minutes), yeast cells are much easier to handle than, for example, tissue cells of mammals. The constructed yeast strains are the basis of a biotechnological analysis and screening system.

### DESCRIPTION OF FIGURES

FIG. 1: Vector pUC18pma1.

FIG. 2: Vector p774.

FIG. 3: Genomically integrated signal potency for cytotoxicity testing.

FIG. 4: Genomically integrated signal potency for genotoxicity testing.

FIG. 5: Checking the integration of

(A) pma1-DsRed1 at the Leu2 locus (oligonucleotides: leu2int_antisense1/pma1-158_antisense; positive: yeast clone Nos. 2, 3, 4) and

(B) egfp-URA3 at the RAD54 locus (oligonucleotides: prerad_sense_int1/ura3_antisense; positive: yeast clone No. 3).

FIG. 6: Photos of the S. cerevisiae yeast strain according to the invention in fluorescence tests of Example 3.3 after 8 hours of incubation with (A) 0.05 ng/ml, (B) 0.5 ng/ml of mitomycin C and (C) 0.1 ng/ml, (D) 0.01 ng/ml of TPhT.

FIG. 7: Growth of the S. cerevisiae yeast strain according to the invention after 8 hours of incubation as a function of the inhibitor concentration in the culture medium.

### DETAILED DESCRIPTION OF THE INVENTION

“Functional” and “functionally linked” within the meaning of the present invention means that the corresponding genes are arranged or integrated into the genome of the yeast host strains in such a way as to be expressed depending on the “switching condition” of the promoter.

“Stably integrated” within the meaning of the present invention means that the corresponding characteristic is always retained in the mitotic proliferation of the yeast strains without external selection pressure, and passed on to the offspring.

In particular, the modified yeast strain according to embodiments (1) and (2) of the invention is a yeast strain of the phylum Ascomycota, more preferably a yeast strain of the order Saccharomycetales, the family Candidaceae or the genus Kluyveromyces. Of these, the yeasts of the order Saccharomycetales, especially those of the family Saccharomycetaceae, are especially preferred. Suitable Saccharomycetaceae are the species Saccharomyces cerevisiae and Saccharomyces uvarum, S. cerevisiae being preferred.

“Different reporter genes” means that the two reporters expressed can be identified and quantified when commonly expressed in the modified yeast strain.

“Different promoters” within the meaning of the present invention means that the promoters employed in the genotox and cytotox cassettes can be independently induced by genotoxic and cytotoxic agents, respectively, and enable the expression of the respective reporter genes functionally linked to them.

The first promoter, which is present in the genotox cassette, is preferably a promoter which is induced by genotoxic agents and controls repair mechanisms which are activated in consequence of primary DNA damage. These include both heterologous promoters, such as the prokaryotic SOS promoter (Y. Oda, Mutat. Res. 147: 219-229 (1985), and G. Reiferscheid et al., Mutat. Res. 253: 215-223 (1991)), and homologous promoters for the regulation of gene or cell repair genes. In the present invention, a homologous promoter, especially a promoter of the RAD genes (such as RAD54, RAD26 and RDH454, wherein the RAD54, RAD26 and RDH454 promoters shown in SEQ ID NOS: 1 to 3 are particularly preferred) or of the heat shock genes (such as HSP70 and HSP82, wherein the HSP70 and HSP82 promoters shown in SEQ ID NOS: 4 and 5 are particularly preferred), is preferably employed in the genotox cassette.

The second promoter present in the cytotox cassette is preferably a promoter which regulates the constitutive expression of household genes and is deactivated by cytotoxic agents. Both heterologous and homologous promoters can be employed. In the present invention, a homologous promoter, especially a promoter of a tubulin (such as α-tubulin promoters including TUB1 and TUB3 promoters, wherein the TUB1 and TUB3 promoters shown in SEQ ID NOS: 6 and 7 are particularly preferred) or of a metabolic enzyme (such as PMA1, PMA2 and H⁺-ATPase promoters, wherein the PMA1 and PMA2 promoters shown in SEQ ID NOS: 8 and 9 are particularly preferred), is preferably employed in the cytotox cassette.

The first and second reporter genes may be any reporter gene, provided that the two reporter genes do not interfere with each other, i.e., can be detected separately. Suitable reporter genes include, for example, fluorescent markers (e.g., the green fluorescent protein (GFP) from Aequoria victoria, the red fluorescent protein, such as from the Indo-Pacific sea anemone species Discosoma, or mutants thereof adapted for the use in yeasts), enzymes (especially those which can be secreted by the yeast and then detected by a color reaction, such as peroxidases, esterases and phosphorylases), or antigens (which can be detected by immunoassays, such as c-myc and Hab). It is particularly preferred to use two non-interfering fluorescent markers.

In a particularly preferred embodiment, the two reporter genes comprise nucleic acid sequences which code for the “green fluorescent” gene from Aequoria victoria or mutants thereof and for the “red fluorescent” gene from the Indo-Pacific sea anemone species Discosoma or a mutant thereof. Particularly preferred are those mutants of the fluorescent proteins which are encoded by the DNA sequences shown in SEQ ID NOS: 10 and 12. The genotox and cytotox cassettes can comprise further functional DNA sequences/genes, such as selection marker genes (also referred to as “selectable markers” hereinafter), which may serve, inter alia, for the selection for successful integration, as well as recombinase recognition sequences and splicing sites which serve for removing undesirable segments in the inserted cassette, such as the selection marker genes.

The selectable markers can be both auxotrophic markers, such as the auxotrophic markers URA3 (see SEQ ID NO: 14) and LEU2, or genes which cause resistance, for example, against G418 (aminoglycoside phosphotransferase gene).

According to embodiment (2) of the yeast strain, one or more of the xenobiotic translocation genes present in the yeast host strain which are necessary for the export of toxic substances have been deleted or disrupted. Such translocation genes (also referred to as “ABC transporter genes”) are summarized in the following Tables 1 and 2.

TABLE 1
The complete yeast genome codes for 29 ABC genes

| | GENE LIBRARY | | | | | | |
|---|---|---|---|---|---|---|---|
| PROTEIN | ACCESSION | CHR | SIZE | TMS | TOPOLOGY | KNOWN FUNCTION | OTHER NAMES |
| ADP1 | X59720 | III | 1049 | 10 | TM-NBD-TM | | YCR011 |
| OR26.01 | X87331 | XV | 1006 | 12 | TM-NBD-TM | | |
| PDR5 | L19922 | XV | 1511 | 14 | (NBD-TM)2 | cycloheximide and multidrug | STS5 |
| | | | | | | resistances | YDR1 |
| PDR10 | Z49821 | XV | 1564 | 14 | (NBD-TM)2 | | YIL013/YIB |
| | | | | | | | Y1329919 |
| PDR11 | Z47047 | IX | 1411 | 15 | (NBD-TM)2 | | LPE14 |
| PDR12 | U39205 | XVI | 1511 | 12 | (NBD-TM)2 | | D950924 |
| PDR15 | U32274 | IV | 1529 | 14 | (NBD-TM)2 | | YD811916 |
| SNQ2 | Z48008 | IV | 1501 | 12 | (NBD-TM)2 | 4-NQO and multidrug resistances | |
| YNR070 | Z71685 | XIV | 1333 | 12 | (NBD-TM)2 | | |
| 01125 | | XV | 1095 | 13 | (NBD-TM)2 | | |
| ATM1 | Z49212 | XIII | 690 | 5 | TM-NBD | preservation of mitochondrial DNA | MDY |
| | | | | | | | YM995203 |
| L1313 | X91488 | XII | 1559 | 17 | (NBD-TM)2 | | |
| MDL1 | U17246 | XII | 696 | 6 | TM-NBD | | |
| MDL2 | L16993 | XVI | 812 | 5 | TM-NBD | | SSH1 |
| STE6 | Z28209 | XI | 1290 | 12 | (NBD-TM)2 | α-factor export | YKL209 |
| YCF1 | Z48179 | IV | 1515 | 17 | (NBD-TM)2 | cadmium und dia resistances | YD930211 |
| YHL035 | U11583 | VIII | 1592 | 16 | (NBD-TM)2 | | |
| YKR103/104 | Z28328 | XI | 1524 | 17 | (NBD-TM)2 | possible pseudogene | |
| YOR1 | | VII | 1477 | 15 | (NBD-TM)2 | oligomycin resistance | |
| L0705 | | XII | 1661 | 20 | (NBD-TM)2 | | |
| GCN20 | D50617 | VI | 752 | 0 | NBD-NBD | interacts with tRNA | YFR009 |
| YEF3 | U20865 | XII | 1044 | 3 | NBD-NBD | stimulation of aminoacyl-tRNA | TLI3EFC1 |
| | | | | | | binding | 196725 |
| YER036 | U18796 | V | 610 | 0 | NBD-NBD | | |
| YDR087 | | IV | 608 | 4 | NBD-NBD | | |
| YNL014 | Z71290 | XIV | 1044 | 3 | NBD-NBD | | |
| RRA1196 | | XVI | 1196 | 3 | NBD-NBD | | |
| PAL1 | L34491 | | 870 | 5 | TM-NBD | oleate oxidation | SSH2 PXA1 |
| YKL185 | Z28188 | XI | 853 | 6 | TM-NBD | possible interaction with PAL1 | YKL741 |

TABLE 2
Classification and illustrative examples of different clinically relevant antibiotic transporters

| | | | | | |
|---|---|---|---|---|---|
| #3.(1-11): | #3.1.(1-70): | #3.1.35. DrugE1 | MsrA S. epidermidis | | |
| Primarily | ABC | Drug Exporter-1 | (ery) | | |
| active | ATP binding | | | | |
| transporters | cassette | | | | |
| | | #3.1.47. DrugE2 | LmrA L. lactis (drugs) | | |
| | | Drug Exporter-2 | | | |
| | | #3.1.61. MDR | | | MDR1 H. sapiens (phosphol.; fq, |
| | | Multi Drug | | | lm, ml, rif, tet) |
| | | Resistance | | | |
| | | #3.1.65 PDR | | Pdr5 S. cerevisiae | |
| | | Pleiotropic Drug | | (azo, chl, ery, lm, tet) | |
| | | Resistance | | Snq2 S. cerevisiae (azo) | |
| | | | | CDR1 C. albicans (azo, chl) | |
| | | | | AtrA, B A. nidulans (ag, azo) | |
| | | #3.1.67 CT1 | | Ycf1 S. cerevisiae (conjugates) | |
| | | Conjugate | | | |
| | | Transporter-1 | | | |
| | | #3.1.68 CT2 | | Yor1 S. cerevisiae (ery, tet) | MRP1-6 H. sapiens |
| | | Conjugate | | | (conjugates, phosphol. fq) |
| | | Transporter-2 | | | |

The xenobiotic translocation genes which are deleted or disrupted include PDR5, YOR1, SNQ2, YCF1, PDR10, PDR11 and PDR12. Preferably, at least the PDR5 gene is deleted, and more preferably, a modified isogenic Saccharomyces cerevisiae yeast host strain having deletions in the PDR5, YOR1 and SNQ2 genes is employed.

The method according to embodiment (3) of the invention comprises the inserting of the cassette into the yeast genome. The yeast transformation can be effected in accordance with the lithium acetate method as described by R. Rothstein in Methods in Enzymology 194: 281-302 (1991). Yeast-genetic methods, especially for Saccharomyces cerevisiae, are in accordance with the method described in F. Sherman et al., Cold Spring Harbor Laboratory, Cold Spring Harbor, N.Y. (1981), which comprises the crossing of the modified strains and isolation of the diploid strains by micromanipulation. The integration is preferably followed by means of the above mentioned selectable markers (auxotrophy and/or resistances). In detail, the cassettes containing markers are introduced, the subsequent crossing results in isogenic strains being obtained and selection of those strains which have a stable integration of the desired cassettes in the yeast genome after transformation (e.g., in the case of the plasmid p774pma1Dsred and the DNA cassette rad54::egfp, growth in culture media and selection of the strains which grow without supplements of leucine and uracil are effected).

To obtain the pdr5yor1snq2 triple-mutant yeast host strain, for example, the Saccharomyces cerevisiae yeast host strain, the genes can be deleted and/or disrupted by introducing one or more selectable markers (auxotrophy and/or resistances).

The selectable biosynthetic marker genes (auxotrophy needs and/or resistances) can be introduced into the loci of the wild type potassium transporter genes by recombinant DNA techniques. Suitable selectable markers are the above mentioned auxotrophy and resistance markers. Such modified alleles can then be transformed into Saccharomyces cerevisiae, where they replace the wild type loci by homologous recombination. The strains comprising modified alleles can be established by selecting for the biosynthetic marker or markers.

In addition, the selectable biosynthetic markers introduced into the loci of the non-specific translocation systems represent a simple route for transferring these mutations into genetically different lines (crossing). A strain which contains a mutation in one of the xenobiotic translocation genes (e.g., PDR5 or YOR1 or SNQ2) can be crossed with a strain of the opposite mating type which bears a mutation in another xenobiotic translocation gene (e.g., PDR5 or YOR1 or SNQ2) to form diploids. By subsequent sporulation to form haploids (tetrad analysis), the isogenic offspring can then be selected for the presence of the biosynthetic markers. The pdr5yor1snq2 triple-mutant Saccharomyces cerevisiae phenotype can be established by growth tests on selective culture media with, for example, ketokonazole concentrations of 100 μM or less. A yeast strain according to the invention can be established by a test in which it is analyzed whether a substance intoxinates the pdr5yor1snq2 triple-mutant Saccharomyces cerevisiae phenotype. Thus, the strain to be tested is incubated with the substance by growth tests on selective culture media. Thus, this simple method in which changes in growth can be observed by agar plate tests and/or in liquid culture can detect specifically active substances which modulate metabolic functions or morphologic changes. For testing different substances or solutions, the screening method can comprise changes such as metabolic activity or reduced growth rate. The test substances which are employed in the method for the detection of specific modulators can be, for example, synthetic or natural products. Natural products comprise vegetable, animal or microbial extracts.

According to embodiment (4), the present invention relates to a method for the detection of environmentally relevant noxious substances, i.e., a method for the dose-dependent genotoxic and cytotoxic substance testing, especially for organotin environmental poisons. Thus, the yeast strains according to the invention are preincubated in a nutrient solution, preferably a YNB nutrient solution (1.7 g/l yeast nitrogen base without amino acids), 5 g/l NH₄SO₄, 2% D-glucose, 0.5 g/l amino acid mix (consisting of 250 mg of adenine, 500 mg of tryptophan, 100 mg of arginine, 100 mg of methionine, 150 mg of tyrosine, 150 mg of lysine, 300 mg of valine, 500 mg of threonine, 500 mg of serine, 250 mg of phenylalanine, 100 mg of asparagine, 10 mg of glutamic acid, 100 mg of histidine) at pH 5.6 to 5.9 at 25 to 35° C., preferably 30° C., for 12 to 18 h with shaking and aerating.

Aliquots of this preincubated solution containing from 1×10⁵ to 1×10⁷ yeast cells per ml are contacted with up to 1% by volume of an aqueous solution of the substance to be tested or up to 0.1% by weight of the substance to be tested as a solid, incubated under the above described preincubation conditions (about 5 to 20 h), and the effect on the reporter potences is detected in the incubated yeast cultures. If required, with a positive result, a comparison can be effected with a reference sample containing a known concentration of noxious substances. The detection is effected specifically for the reporters present in the modified yeast strain. For a reporter system comprising the above mentioned “green” and “red fluorescent proteins”, detection is effected by measuring the fluorescence intensity at 508 and 583 nm. A detailed description for a test with a yeast strain using such a reporter system is given in Example 4.

Further, the present invention relates to a test kit and a biosensor (also referred to as “biotest” hereinafter) comprising the genetically modified yeast cells according to the invention, especially Saccharomyces cerevisiae cells. This biotest is easy to handle and represents a low-expenditure detection method for establishing genotoxic and cytotoxic effects of complex mixtures of substances in aqueous solutions. Sterile working is not required. The constructed hypersensitive yeast strain with genotoxic and cytotoxic signaling can be employed as a biotechnological high-throughput test system for the concentration-dependent detection of complex environmental contaminations as well as, in particular, organotin compounds in solutions.

This technology has a broad range of applications for the detection of environmentally relevant noxious substances in health care for humans:

1. as an early warning system in water surveillance;

2. for the ecotoxicological evaluation of waste waters;

3. as a biotest in ecotoxicology;

4. for the functional monitoring of sewage treatment plants;

5. in medicine, for the toxicity screening of medicaments and substances; and

6. in the industry, for monitoring the solutions used in the production process.

The advantages of the test method according to the invention consist in the following:

i) the sensitivity with which the active material can be identified;

ii) the number of samples which can be tested; and

iii) the short time (8 hours) in which the test can be performed;

iv) the non-sterile performance of the test; and

v) the possible parallel detection of genotoxic and cytotoxic effects by differentiated signals.

The biotechnological usefulness of well growing yeast strains stably detecting genotoxicity and cytotoxicity consists in the early detection of noxious environmental loads and, for estimating the risk for human health, i) as an early warning system in water surveillance, ii) for the ecotoxicological evaluation of waste waters, iii) as a biotest in ecotoxicology, iv) for the functional monitoring of sewage treatment plants, v) in medicine, for the toxicity screening of medicaments and substances, and vi) in the industry, for monitoring the solutions used in the production process, since yeasts can be used in growth-based serial tests for the screening of many different test solutions on a very small scale and with a high efficiency (screening methods in microtitration dishes).

Finally, the present invention relates to a method for the detection of specific modulators of the expressed reporter genes, for example, the pma1-Dsred1 and rad54::egfp reporter genes, comprising:

(a) the treatment of a yeast host strain, especially a Saccharomyces cerevisiae yeast host strain, in which receptor and reporter signal potencies for genotoxic and/or cytotoxic environmental contaminations are stably integrated in the genome as defined above, but the PDR5, SNQ2 or YOR1 xenobiotic translocation systems of the yeast Saccharomyces cerevisiae are not expressed, with test substances;

(b) determinations of growth in the presence or after the application of a test substance; and

(c) measurements of the increase or decrease of reporter gene expression, for example, the fluorescent intensity of this strain, in the presence or after the application of a test substance.

The following Examples further illustrate the invention.

### EXAMPLES

### General Methods

Recombinant DNA technology: For the enrichment and manipulation of DNA, standard methods were employed as described in J. Sambrook et al., Cold Spring Harbor Laboratory Press, Cold Spring Harbor, N.Y. (1989). The molecular-biological reagents used were employed according to the manufacturer's instructions.

Yeast transformation: Saccharomyces cerevisiae strains were transformed in accordance with the lithium acetate method as described by R. Rothstein in Methods in Enzymology 194: 281-302 (1991).

Yeast-genetic methods: Saccharomyces cerevisiae strains were crossed in accordance with the method described in F. Sherman et al., Cold Spring Harbor Laboratory, Cold Spring Harbor, N.Y. (1981), and diploid strains were isolated by micromanipulation.

Primers: Gene library Accession SCYGL163C, 4837 bp, chromosome VII, reading frame YGL163c, deposited on Aug. 11, 1997:

Oligonucleotide postrad_sense (SEQ ID NO:17):

5′ GAG AGC TAG CAG ACT CGA GCT CTT ACA TAC ATG TAC TTA TAA AAC 3′,

positions 380-406 in the genomic sequence; the nucleotides added for reasons of cloning technology are represented in italics, and the nucleotides inserted to obtain an XhoI restriction site are underlined.

Oligonucleotide postrad_antisense (SEQ ID NO:18)

5′ GAG AGG TAC CAG TTA AAG TTA ATC CTT CTG AGA G 3′,

positions 18-51 in the genomic sequence; the nucleotides added for reasons of cloning technology are represented in italics, and the nucleotides inserted to obtain an KpnI restriction site are underlined.

Oligonucleotide prerad_sense SEQ ID NO:19):

5′ GAG AGC GGC CGC CTC ATA CTC GAG GGA AAT TCG 3′,

positions 4378-5357 in the genomic sequence; the nucleotides added for reasons of cloning technology are represented in italics, and the nucleotides inserted to obtain an NotI restriction site are underlined.

Oligonucleotide prerad_antisense (SEQ ID NO:20):

5′ GAG AGG ATC CGG TAA TCT GCG TCT TGC CAT CAG 3′,

positions 3084-3106 in the genomic sequence; the nucleotides added for reasons of cloning technology are represented in italics, the nucleotides inserted to obtain an BamHI restriction site are underlined, and the start codon of the RAD54 gene (as an inverse complement) is represented in boldface.

Oligonucleotide pma1-158_antisense (SEQ ID NO:21):

5° CGG CTG GTT CTA 3′,

corresponds to positions 170-157 in the 939 bp deposited DNA sequence of the “promotor binding protein” gene (Gene library accession YSCHATPASA, M25502).

Oligonucleotide LEU2int_antisense (SEQ ID NO:22):

5′ GTC GAC TAC GTC GTT AAG GCC G 3′,

corresponds to positions 92511-92489 in the 316613 bp deposited DNA sequence of the LEU2 gene (Gene library accession NC_{—}001135, GI:10383748, Saccharomyces cerevisiae chromosome III, complete chromosome sequence).

Oligonucleotide prerad_sense_int1 (SEQ ID NO:23):

5′ ACA AAG CTC CTC TCC TGC TCA AG 3′,

positions 4503-4481 in the RAD54 gene sequence (Gene library accession SCYGL163C, Z72685, Y13135),

Oligonucleotide ura3_antisense (SEQ ID NO:24):

5′ ACT AGG ATG AGT AGC AGC ACG T 3′,

positions 267-245 in the URA3 gene sequence (Gene library accession 406851).

### Example 1

### Construction of the Dsred Integration Plasmid for Cytotoxic Signaling

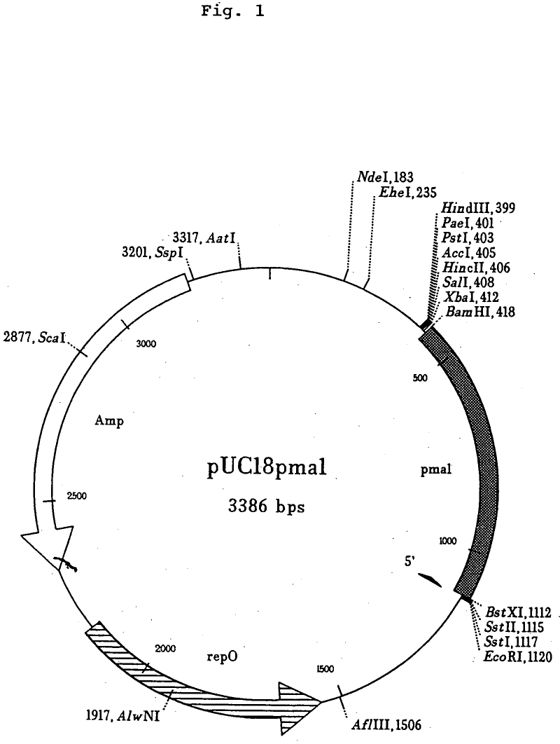

Construction of the plasmid p774pma1Dsred: For the transcription of the “red fluorescent protein” gene in the yeast Saccharomyces cerevisiae, the yeast promoter of the plasma membrane ATPase PMA1 was used. The pma1 promoter, which is constitutively active in Saccharomyces cerevisiae, was isolated as EcoRI/BamHI 0.93 kb fragment from the plasmid pRS408 (obtained from Dr. A. Goffeau, Université Catholique de Louvain-la-Neuve, Belgium) after separation by agarose gel electrophoresis. In a ligation, the 0.93 kb pmal EcoRI/BamHI fragment was ligated with the EcoRI/BamHI-restricted plasmid vector pUC18. The obtained plasmid pUC18-pma1 (see FIG. 1) was confirmed by restriction mapping and sequencing.

From the plasmid pDsRed1-N1 (Clontech; SEQ ID NO:16), the “red fluorescent protein” gene (start codon 679-681, stop codon 1357-1359) was cleaved at position 1361 with the restriction endonuclease NotI, the linearized plasmid was separated as a 4.7 kb fragment in an agarose gel electrophoresis and eluted from the gel matrix. The sticky ends obtained from the restriction of the NotI site were filled in a subsequent DNA polymerase enzyme reaction (Klenow fragment) with 0.1 mM free nucleotides (dNTPs) in 5′→3′ direction to obtain blunt ends. In a further step, the linear 4.7 kb pDsRed1-N1 fragment was cleaved with the restriction endonuclease BamHI at position 661. By agarose gel electrophoresis, the BamHI(NotI filled) 0.7 kb fragment with the “red fluorescent protein” gene was separated and eluted from the gel matrix. This fragment was ligated with the pUC18-pma1 BamHI/HincII restricted vector, transformed into bacteria (E. coli XL1 Blue, Stratagene), and the colonies obtained after incubation at 37° C. were analyzed. By the sequencing and EcoRI/HindIII restriction mapping of isolated plasmid DNA and subsequent separation in agarose gel electrophoresis, the 1.63 kb pma1-DsRed1 composite fragment was confirmed.

The pUC18-pma1-Ds-Red1 plasmid was cleaved with the restriction endonuclease SacI in the polylinker region upstream from the combined pma1-DsRed1 fragment, separated as a linear 4.31 kb fragment in an agarose gel electrophoresis and eluted from the gel matrix. The sticky ends obtained from the restriction of the SacI site were filled in a subsequent DNA polymerase enzyme reaction (Klenow fragment) with 0.1 mM free nucleotides (dNTPs) in 5′→3′ direction to obtain blunt ends. In a further step, the linear 4.31 kb pUC18-pma1-Ds-Red1 fragment was cleaved with the restriction endonuclease HindIII in the polylinker region upstream from the combined pma1-DsRed1 fragment, which was separated by agarose gel electrophoresis and isolated from the gel matrix as a 1.63 kb fragment.

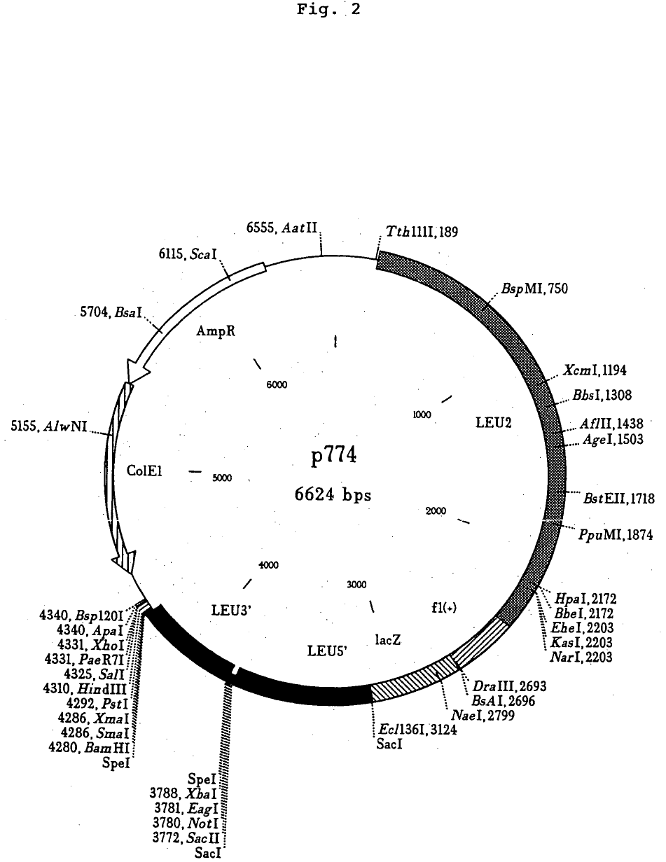

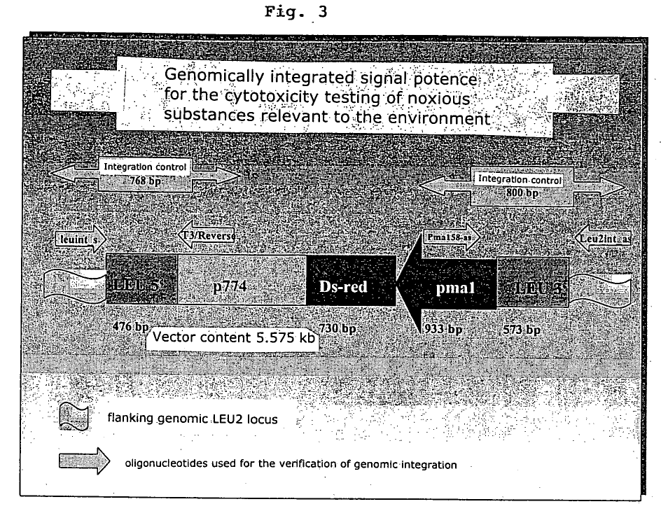

The plasmid p774 (Connelly & Heiter (1996) Cell 86, 275-285; obtained from Dr. P. Ljungdahl, Ludwig Institute of Cancer Research, Stockholm, Sweden; see FIG. 2) was cleaved with the restriction endonucleases SmaI/HindIII. This linear 6.6 kb vector was ligated with the 1.63 kb pma1-Ds-Red1 composite fragment, transformed into bacteria, and the colonies obtained after incubation at 37° C. were analyzed. By a BamHI/SalI restriction mapping of isolated plasmid DNA and subsequent separation in agarose gel electrophoresis, the cloning of the combined 1.63 kb pma1-DsRed1 composite fragment into p774 was confirmed by obtaining three fragments (6.6 kb p774, 0.93 kb pma1 promoter, 0.7 kb “red fluorescent protein” gene). FIG. 3 shows a schematic representation of the plasmid construct which was used for the integration of the cytotoxic signaling. Using the vector p774-pma1-DsRed1, the “red fluorescent protein” gene was stably integrated into the gene locus for the biosynthetic marker LEU2 of the Saccharomyces cerevisiae yeast host strain under the control of the yeast ATPase pma1 promoter.

### Example 2

### Construction of the Rad54/gfp//URA3 Integration Cassette for Genotoxic Signaling

Construction of the plasmid pBSK-rad54-gfp-URA3: For the integration of the genotoxic signaling, a 1.841 kb DNA fragment consisting of the gene for the “green fluorescent protein” in the “yeast enhanced version” egfp with a subsequent selectable biosynthetic Saccharomyces cerevisiae URA3 marker gene coding for the orotidine-5′-phosphate decarboxylase was first isolated from the plasmid pBSK-tok-egfp-URA3 (obtained from Dr. Jost Ludwig, MNF, Tübingen University) after cleaving with the restriction endonucleases BamHI and XhoI and separation by agarose gel electrophoresis. This fragment was ligated with the BamHI/XhoI-restricted plasmid vector pBSKII (Stratagene), transformed into bacteria (E. coli XL1 Blue (Stratagene)), and the colonies obtained after incubation at 37° C. on LB-Amp (Luria-Bertani medium, 100 μg/ml ampicillin) agar plates were analyzed. By BamHI/XhoI-restriction mapping of isolated plasmid DNA, the inserted egfp-URA3 fragment (1.84 kb) in pBSKII (2.96 kb) was confirmed.

From the Saccharomyces cerevisiae wild strain S288C (ATCC, USA), genomic DNA was isolated with standard methods. 20 pg of this chromosomal DNA was employed for a polymerase chain reaction (PCR) with DNA polymerase from Thermophilus aquaticus (MBI Fermentas) and the oligonucleotide primers postrad_sense and postrad_antisense for the amplification of the 3′-non-coding region of the S. cerevisiae RAD54 gene.

The DNA amplified by the PCR was separated by agarose gel electrophoresis and isolated from the gel matrix as a 0.4 kb fragment. This DNA was cleaved with the restriction endonucleases KpnI/XhoI, separated by agarose gel electrophoresis, isolated from the gel matrix and ligated with the KpnI/XhoI-linearized plasmid vector pBSK-egfp-URA3 (4.82 kb). After transformation into bacteria (E. coli XL1 Blue, Stratagene) and incubation at 37° C. on LB-Amp (Luria-Bertani medium, 100 μg/ml ampicillin [J. Sambrook, E. F. Fritsch and T. Maniatis (1989), In: Molecular cloning: A laboratory manual. Cold Spring Harbor Laboratory Press, Cold Spring Harbor, N.Y.]) agar plates, the colonies obtained were analyzed. By a XhoI/KpnI restriction mapping of isolated plasmid DNA and separation in an agarose gel, the 2.26 kb egfp-URA3-postrad54 composite fragment in pBSKII (2.96 kb) was confirmed.

From the Saccharomyces cerevisiae wild strain S288C (ATCC, USA), genomic DNA was isolated with standard method. 20 pg of this chromosomal DNA was employed for a polymerase chain reaction (PCR) with DNA polymerase from Thermophilus aquaticus (MBI Fermentas) and the oligonucleotide primers prerad_sense and prerad_antisense for the amplification of the 5′-non-coding region of the S. cerevisiae RAD54 gene plus the start codon and a further one.

The DNA fragment amplified by the PCR was separated by agarose gel electrophoresis and isolated from the gel matrix as a 1.3 kb fragment. This DNA was cleaved with the restriction endonucleases BamHI/NotI, separated by agarose gel electrophoresis, isolated from the gel matrix and ligated with the BamHI/NotI-linearized 5.2 kb plasmid vector pBSK-egfp-URA3-postrad54. After transformation into bacteria (E. coli XL1 Blue, Stratagene) and incubation at 37° C. on LB-Amp (Luria-Bertani medium, 100 μg/ml ampicillin) agar plates, the colonies obtained were analyzed. By a NotI/KpnI restriction mapping of isolated plasmid DNA and separation in an agarose gel, the 3.5 kb prerad54-egfp-URA3-postrad54 composite fragment in pBSKII (2.96 kb) was confirmed.

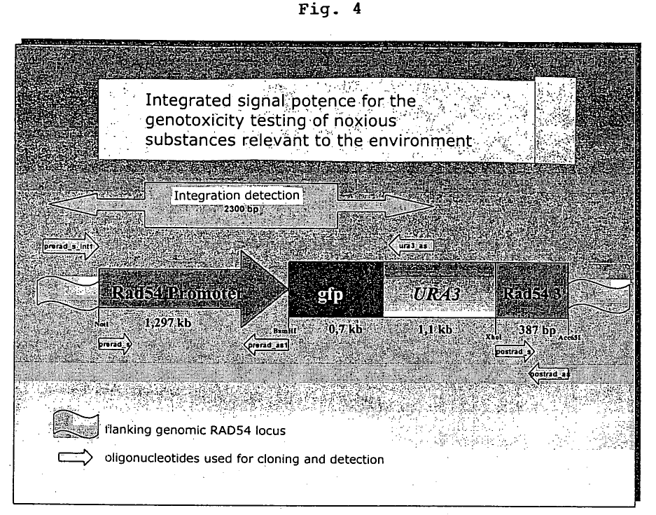

FIG. 4 shows a schematic representation of the prerad54-egfp-URA3-postrad54 DNA construct used for the integration into the S. cerevisiae genome. Using the rad54 non-coding sequences of this cassette, the egfp-URA3 composite fragment was stably integrated in a well-aimed way by homologous recombination into the gene locus of the S. cerevisiae RAD54 gene on chromosome VII. Thus, the entire reading frame coding for RAD54 has been replaced by the egfp-URA3 composite fragment. The RAD54 promoter control elements unchanged by genetic engineering regulate the expression of the “green fluorescent protein” egfp gene.

### Example 3

### Construction of the Saccharomyces cerevisiae Strain with Integrated Cytotoxic and Genotoxic Signaling

3.1. Construction of the Saccharomyces cerevisiae strain expressing pma1-DsRed1 and rad54-gfp: In the plasmid p774-pma1-DsRed1 (see Example 1, FIG. 3), 5′ 476 bp and 3′ 573 bp are inserted as flanking regions of the LEU2 gene for the selective homologous recombination of the cloned insert into the chromosomal locus of the LEU2 gene (chromosome III) in Saccharomyces cerevisiae. For the selective integration of the overall plasmid including the 1.63 kb pma1-DsRed1 composite fragment at the chromosomal locus of the LEU2, gene, a linearization of the plasmid p774-pma1-DsRed1 was introduced with the restriction endonuclease NotI at position 3780 (based on the original plasmid, see FIG. 2) between the flanking LEU2 regions.

After separation by agarose gel electrophoresis and isolation from the gel matrix, the linear 8.25 kb NotI DNA fragment obtained was used for the transformation of the pdr5yor1snq2 triple-mutant Saccharomyces cerevisiae yeast strain, in which the main yeast ABC transporter genes for xenobiotic translocation systems responsible for endogenous resistance have been deleted. Thus, colonies derived from single cells (yeast transformants) were selected for the biosynthetic marker LEU2 which is also contained in the plasmid p774 (LEU2⁺ protrophy).

The transformation of the pdr5yor1snq2 triple-mutant Saccharomyces cerevisiae yeast strain with the DsRed1 gene under the control of the pma1 promoter resulted in the isolation of a Saccharomyces cerevisiae yeast host strain in which the main yeast ABC transporter genes for xenobiotic translocation systems responsible for endogenous resistance are specifically deleted and the DsRed1 gene is stably integrated at the chromosomal locus of the biosynthetic LEU2 gene and expressed under the control of the yeast pma1 promoter (genotype MATa ura3-52 trp1-Δ63 leu2-Δ1 his3-Δ200 GAL2⁺ pdr5-Δ1::hisG snq2::hisG yor1-1::hisG leu2::pma1-DsRed1 LEU2). In cells proliferating by mitosis, the construct for cytotoxic signaling is transmitted to the offspring. After growing a yeast culture, the expression of the DsRed1 gene becomes visible through a red fluorescence with an emission maximum at 583 nm upon spectral excitation at 558 nm.

In the plasmid pBSKII-prerad54-egfp-URA3-postrad54 (see Example 2, FIG. 4), 5′ 1.3 kb flanking prerad54 and 3′ 0.4 kb flanking postrad54 regions are inserted into S. cerevisiae for the selective homologous recombination of the cloned insert egfp-URA3 at the chromosomal locus of the RAD54 gene (chromosome VII). For the selective integration of the prerad54-egfp-URA3-postrad54 cassette at the chromosomal locus of the RAD54 gene, a 3.62 kb fragment was cleaved from the plasmid pBSKII-prerad54-egfp-URA3-postrad54 using the restriction endonucleases NotI/PvuII.

After separation by agarose gel electrophoresis and isolation from the gel matrix, the 3.62 kb prerad54-egfp-URA3-postrad54 fragment obtained was used for the transformation of the yeast strain with a cytotoxic signal potence (genotype MATa ura3-52 trp1-Δ63 leu2-Δ1 his3-Δ200 GAL2⁺ pdr5-Δ1::hisG snq2::hisG yor1-1::hisG leu2::pma1-DsRed1 LEU2). Thus, colonies derived from single cells (yeast transformants) were selected for the biosynthetic marker URA3 contained in the cassette (URA3⁺ protrophy).

The transformation of the pdr5yor1snq2 triple-mutant Saccharomyces cerevisiae yeast strain with an integrated DsRed1 gene under the control of the pma1 promoter with the prerad54-egfp-URA3-postrad54 cassette resulted in the isolation of the Saccharomyces cerevisiae yeast host strain HLY5RG-12B2 in which:

1. the main yeast ABC transporter genes for xenobiotic translocation systems responsible for endogenous resistance have been specifically deleted; and

2. the DsRed1 gene is stably integrated at the chromosomal locus of the biosynthetic LEU2 gene and expressed under the control of the yeast pma1 promoter for cytotoxic signaling; and

3. the egfp gene is stably integrated at the chromosomal locus of the RAD 54b gene and expressed under the control of the yeast rad54 promoter for genotoxic signaling (genotype MATa ura3-52 trp1-Δ63 leu2-Δ1 his3-Δ200 GAL2⁺ pdr5-Δ1::hisG snq2::hisG yor1-1::hisG leu2::pma1-DsRed1 LEU2 rad54::egfp-URA3). Instead of the RAD54 gene, the egfp gene is exclusively expressed in this Saccharomyces cerevisiae strain. In cells proliferating by mitosis, both constructs for cytotoxic and genotoxic signaling are transmitted to the offspring. After growing a yeast culture and induction of the biochemical “RAD54”-mediated signal transduction cascade, the expression of the egfp gene becomes visible through a green fluorescence with an emission maximum at 508 nm upon spectral excitation at 488 nm.

3.2 Characterization of the Saccharomyces cerevisiae strain expressing pma1-DsRed1 and rad54-gfp by PCR analysis: The correct integration of the p774-pma1-DsRed1 plasmid and the egfp-URA3 cassette at the respective chromosomal LEU2 or RAD54 locus of the Saccharomyces cerevisiae genome, genotype MATa ura3-52 trp1-Δ63 leu2-Δ1 his3-Δ200 GAL2⁺ pdr5-Δ1::hisG snq2::hisG yor1-1::hisG leu2::pma1-DsRed1 LEU2 rad54::egfp-URA3, was detected with PCR analyses.

As a template, 20 pg of the genomic DNA from four different selected yeast single colonies, isolated with standard methods, was employed.

The correct integration of the p774-pma1-DsRed1 plasmid at the chromosomal LEU2 locus was detected with an oligonucleotide which is complementary to the coding strand of the inserted pma1 promoter in “sense” direction and an oligonucleotide which is complementary to the coding strand downstream and outside the inserted flanking LEU2 3′ region in “antisense” direction (primers pma1-158_antisense and Leu2int_antisense).

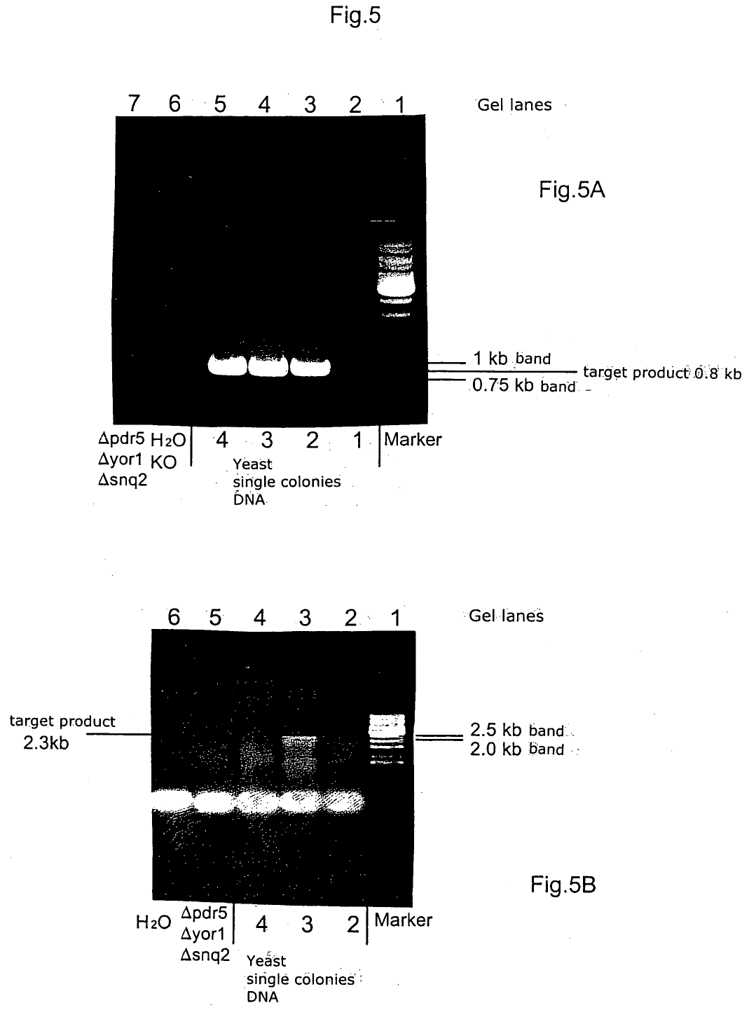

A 0.8 kb specific DNA fragment was to be amplified thereby. For controlling the reaction mixture, a reaction was performed without a template (water control, gel lane 7 in FIG. 5A). For controlling the correct amplification product, 20 pg of the pdr5yor1snq2 triple-mutant Saccharomyces cerevisiae starting strain was employed (negative control, gel lane 6 in FIG. 5A). In FIG. 5A, the reaction mixtures with the isolated DNA from four different yeast single colonies have been separated by agarose gel electrophoresis. Gel lane 1 contains the molecular weight marker (No. VII, MBI Fermentas), gel lane 2 contains the reaction mixture of the analyzed yeast clone 1 in which no specific amplification can be seen, gel lanes 3 to 5 show the reaction mixtures of the analyzed yeast clones 2, 3 and 4 which show the specific amplification of the desired target product of 0.8 kb and thus confirm the successful integration of the p774-pma1-DsRed1 plasmid at the chromosomal LEU2 locus in the yeast single colonies 2, 3 and 4.

The correct integration of the egfp-URA3 cassette at the chromosomal RAD54 locus of the Saccharomyces cerevisiae genome was detected with the DNA of the previously confirmed yeast clones 2, 3 and 4 as templates and with an oligonucleotide which is complementary to the coding strand of the biosynthetic marker gene URA3 in “antisense” direction and an oligonucleotide which is homologous with the coding strand upstream and outside the inserted flanking RAD54 5′ region in “sense” direction (primers prerad_sense_int1 and ura3_antisense).

A 2.3 kb specific DNA fragment was to be amplified thereby. For controlling the reaction mixture, a reaction was performed without a template (water control, gel lane 6 in FIG. 5B). For controlling the correct amplification product, 20 pg of the pdr5yor1snq2 triple-mutant Saccharomyces cerevisiae starting strain was employed (negative control, gel lane 5 in FIG. 5B). In FIG. 5B, the reaction mixtures with the isolated DNA from the previously confirmed yeast colonies 2, 3 and 4 have been separated by agarose gel electrophoresis. Gel lane 1 contains the molecular weight marker (No. VII, MBI Fermentas), gel lanes 2 to 4 contain the reaction mixtures of the analyzed yeast clones 2, 3 and 4, of which only the yeast clone 3 shows a specific amplification of the desired target product of 2.3 kb and thus confirms the successful integration of the egfp-URA3 cassette at the chromosomal RAD54 locus of the Saccharomyces cerevisiae genome of yeast clone 3.

This isolated yeast single colony with the genotype MATa ura3-52 trp1-Δ63 leu2-Δ1 his3-Δ200 GAL2+pdr5-Δ1::hisG snq2::hisG yor1-1::hisG leu2::pma1 -DsRed1 LEU2 rad54::egfp-URA3 was given the designation HLY5RG-12B2 and was deposited on Dec. 5, 2000, as a glycerol culture with the DSMZ—Deutsche Sammlung von Mikroorganismen und Zellstrukturen GmbH, Mascheroder Weg 1b, 38124 Braunschweig, Germany, according to the provisions of the Budapest Treaty under the designation DSM 13954.

3.3 Characterization of the Saccharomyces cerevisiae strain expressing DsRed1 and egfp by growth and fluorescence tests in the presence of inhibitors:

Cultures of Saccharomyces cerevisiae wild strain, of the pdr5yor1snq2 triple-mutant yeast strain and of the genotoxically and cytotoxically signaling strain HLY5RG-12B2 expressing Dsred and egfp were grown in complete medium YPD (2% yeast extract, 1% peptone) with 2% D-glucose, pH 5.0, at 30° C. over night with shaking.

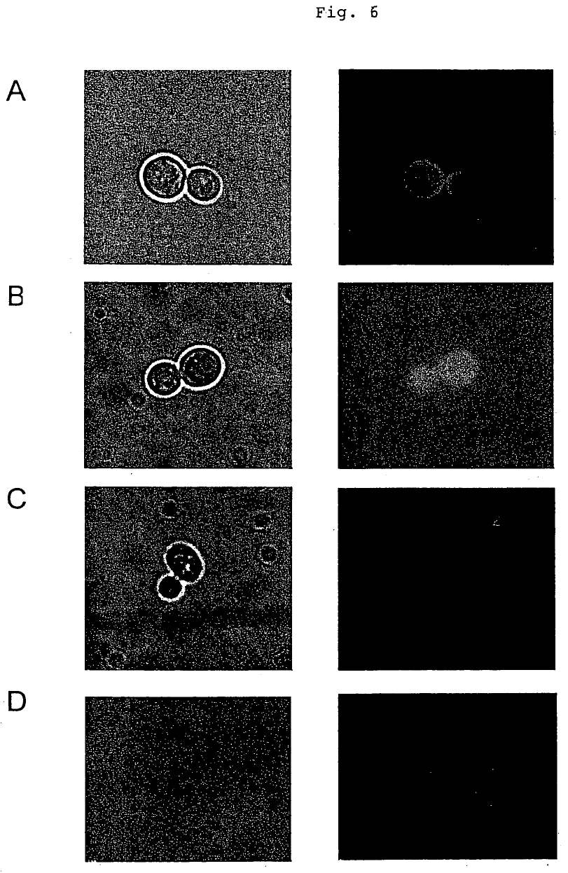

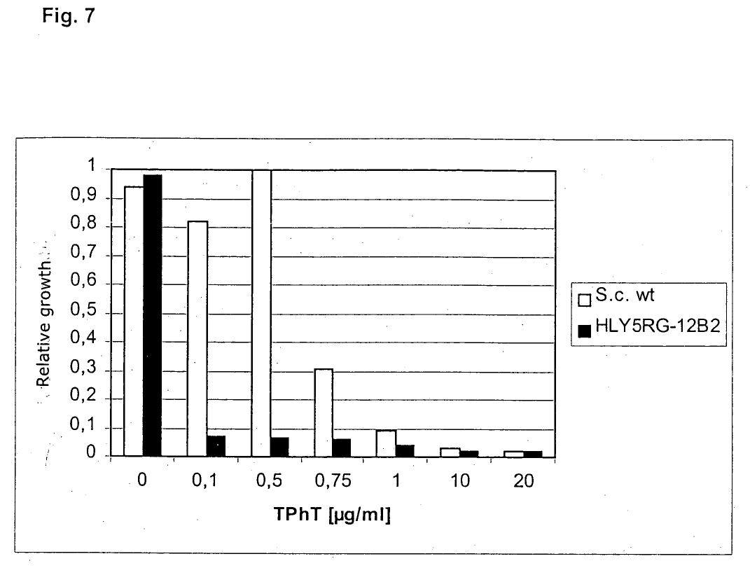

Of each strain, 1×10⁵ cells were incubated under selective and inhibitory conditions (0.67% yeast nitrogen base without amino acids, 0.5% NH₄SO₄, 2% D-glucose, pH 5.0, base medium, for the pdr5yor1snq2 triple-mutant yeast strain supplemented with leucine, tryptophan, histidine and uracil, each 20 μg/l, for the yeast strain HLY5RG-12B2 supplemented with tryptophan and histidine, each 20 μg/l) with 0.05 ng/ml and 0.5 ng/ml mitomycin C as well as 0.01 ng/ml and 0.1 ng/ml TPhT (triphenyltin) at 30° C. for 8 hours. In FIG. 6, fluorescence signals from cells of the constructed and isolated cytotoxically and genotoxically signaling yeast strain HLY5RG-12B2 are shown as a function of inhibitor concentration in the culture medium. FIG. 7 shows the growth of S. cerevisiae wild type cells and of cells of the constructed yeast strain HLY5RG-12B2 after 8 hours of incubation as a function of inhibitor concentration.

Under the above mentioned conditions, the Saccharomyces cerevisiae wild strain grows under inhibitory conditions of 0.05 ng/ml and 0.5 ng/ml mitomycin C as well as 0.01 ng/ml and 0.1 ng/ml TPhT with normal growth rates (doubling time 90 min) without showing a specific red (cytotoxic potential) or green (genotoxic potential) fluorescence at emission maxima of 583 nm. Due to the normal growth rates, this wild strain exhibits a non-specific background fluorescence by stationary cells. The yeast strain having defects in three xenobiotic translocation systems PDR5, YOR1 and SNQ2 (pdr5yor1snq2, triple-mutant) grows with lower growth rates (doubling time 180 min) without showing a specific red (cytotoxic potential) or green (genotoxic potential) fluorescence at emission maxima of 583 nm or 508 nm, respectively. Depending on the used concentration of the genotoxic inhibitor mitomycin C (genotoxic potential, FIGS. 6 A and B), an increasing specific green fluorescence with emission maxima of 508 nm was detected, but no increasing red fluorescence with emission maxima of 583 nm was detected. Depending on the used concentration of the cytotoxic inhibitor TPhT (cytotoxic potential, FIG. 6, C and D), an increasing specific red fluorescence with emission maxima of 583 nm was detected, but no increasing green fluorescence with emission maxima of 508 nm was detected.

Thus, the selective detection of the cytotoxic and genotoxic potential of different substances was confirmed in the S. cerevisiae strain HLY5RG-12B2.

### Example 4

### Cytotox and Genotox Test Methods Using HLY5R

For cytotoxic and genotoxic substance testing, a liquid preculture of the Saccharomyces cerevisiae yeast strain HLY5RG-12B2 was grown in a 5 ml volume consisting of YNB medium (1.7 g/l yeast nitrogen base without amino acids), 5 g/l NH₄SO₄, 2% D-glucose, 0,5 g/l amino acid mix (consisting of: 250 mg of adenine, 500 mg of tryptophan, 100 mg of arginine, 100 mg of methionine, 150 mg of tyrosine, 150 mg of lysine, 300 mg of valine, 500 mg of threonine, 500 mg of serine, 250 mg of phenylalanine, 100 mg of asparagine, 10 mg of glutamic acid, 100 mg of histidine), pH 5.9, at 30° C. over night (12 to 18 hours) with shaking (180 rpm). The cells were then in a logarithmic phase of growth; an aliquot was tested by microscopy and the number of cells counted.

Of this precultured strain, 1×10⁵ cells/ml were inoculated for the test in the same medium. For the genotoxic substance tests, serial concentrations for calibration were prepared with four different concentrations of mitomycin C, beginning with 0.05 ng/ml and ending with 0.5 ng/ml. For the cytotoxic substance tests, serial concentrations for calibration were prepared with four different concentrations of TPhT (triphenyltin), beginning with 0.01 ng/ml and ending with 0.1 ng/ml.

Of aqueous solutions to be tested, dilutions were prepared in descending concentrations in steps of ten in a suitable solvent (at least 10 per solution to be tested) and added to the cells provided in culturing tubes (1 to 10 ml) or microtitration wells (50 to 200 μl).

Of solid substances to be tested, defined amounts were weighed (ng to μg to mg range) and added to the cells provided in culturing tubes (1 to 10 ml) or microtitration wells (50 to 200 μl), or defined solutions (in percent or in g/l) were prepared in a suitable solvent and also added to the cell suspension provided.

For control, a number of culturing tubes (1 to 10 ml) or microtitration wells (50 to 200 μl) with cells provided therein were pipetted without any substance or solution to be tested.

All culturing tubes (1 to 10 ml) or microtitration wells (50 to 200 μl) thus prepared were incubated for 8 hours at 30° C. with shaking (180 rpm).

Subsequently, a spectral excitation at 489 nm was performed for the genotoxic substance testing, and the intensity of the emission maxima at 508 nm (specific of egfp) was measured for all cell suspensions. These data were recorded and evaluated, including the corresponding calibration series and the control series.

For the cytotoxic substance testing, a spectral excitation at 558 nm was performed, and the intensity of the emission maxima at 583 nm (specific of DsRedp) was measured for all cell suspensions. In addition, a determination of the simple growth of the cells by measuring the optical density at 600 nm was performed. These data were recorded and evaluated, including the corresponding calibration series and the control series.

1. A modified yeast strain in which
(1) a genotox cassette comprising a first promoter inducible by genotoxic agents and a first reporter gene functionally linked to the first promoter; and
(b) a cytotox cassette comprising a second promoter inducible by cytotoxic agents and a second reporter gene functionally linked to the second promoter;
wherein the promoters and reporter genes in (1) and (2) are respectively distinct from each other;
are stably and functionally integrated in the genome of a yeast host strain.

2. The yeast strain according to claim 1, wherein said yeast host strain is of the phylum Ascomycota, especially a Saccharomyces cerevisiae strain.

3. The yeast strain according to claim 1 or 2, wherein said yeast strain has been sensitized by disrupting or deleting one or more of the xenobiotic translocation genes present in the yeast host strain.

4. The yeast strain according to claim 1, wherein said xenobiotic translocation genes are selected from PDR5, YOR1, SNQ2, YCF1, PDR10, PDR11 and PDR12, especially wherein at least the PDR5 gene is deleted, and more preferably, the PDR5, YOR1 and SNQ2 genes are deleted.

5. The yeast strain according to one or more of claims 1 to 4, wherein:
(i) said first promoter is a promoter which is induced by genotoxic agents and controls repair mechanisms which are activated in consequence of primary DNA damage, preferably a promoter for the regulation of gene or cell repair genes, more preferably a promoter of the Rad genes or the heat shock genes; and/or
(ii) said second promoter is a promoter which regulates the constitutive expression of household genes and is deactivated by cytotoxic agents, especially a promoter of a tubulin or of a metabolic enzyme; and/or
(iii) said first and second reporter genes are selected from fluorescent markers, enzymes or antigens, especially being two non-interfering fluorescent markers.

6. The yeast strain according to claim 1, wherein said first promoter is a Rad54 promoter, said first reporter gene is a green fluorescent protein, especially GFP from Aequoria victoria or a mutant thereof, said second promoter is a Leu2 promoter, and said second reporter gene is a red fluorescent protein, especially Dsred from Discosoma or a mutant thereof.

7. The yeast strain according to one or more of claims 1 to 6, wherein the genotox cassette and/or the cytotox cassette further contain functional DNA sequences or functional genes, especially selectable marker genes, recombinase recognition sequences and/or splicing sites.

8. The yeast strain according to claim 1, wherein said yeast strain is a Saccharomyces cerevisiae mutant pdr5yor1snq2 LEU2::pma1-Dsred RAD54::gfp, especially HLY5RG-12B2 (DSM 13954).

9. A method for the preparation of a modified yeast strain according to claims 1 to 8, comprising the integration of genotox and cytotox cassettes into a yeast host strain.

10. The method according to claim 9, wherein one or more xenobiotic translocation genes are further disrupted or deleted.

11. The method according to claim 10, wherein said genotox and cytotox cassettes are integrated into the genome of a Saccharomyces cerevisiae pdr5yor1snq2 yeast host strain.

12. A method for the detection of noxious substances relevant to the environment, comprising:
(a) the treatment of a modified yeast strain according to claims 1 to 8 with a test substance or a mixture of test substances;
(b) determinations of growth in the presence or after completion of the treatment with said test substance/mixture of test substances; and
(c) measurements of the increase or decrease of the reporter gene activity of the yeast strain in the presence or after completion of the treatment with said test substance/mixture of test substances.

13. The method according to claim 12, which is suitable for the detection of genotoxic substances and/or cytotoxic substances.

14. Use of a modified yeast strain according to claims 1 to 8 for testing the genotoxicity and/or cytotoxicity of complex environmental contaminations.

15. A test kit and biosensor for testing the genotoxicity and/or cytotoxicity of complex environmental contaminations, comprising a modified yeast strain according to claims 1 to 8.


SEQUENCE LISTING (24 sequences)

SEQ ID NO: 1 - DNA, 1275 bp, Saccharomyces cerevisiae
  promoter (1)..(1275)
cagttataaggaaatatatatggtaccttgaaatagagctaagttctgagcgtttgatttttctatactttcgtagcagctttagtgaagaaagagaaaagttgctgctctagattttgtatcggctattacgtcaagtgagaagagttttggccttcgcttcagttagatcttctattatttccttttttttctttttgtttgtaattgttttttatttttttttttgcgcgaaactttgctatattgggtaacgcgtaaaatactttttattattgcagtaaggcggaagggtcttcccctttgcatgttaaatagcatacatggcaccactcaggtccagaacgtgacacatctttgcaccacttgtgtattttcaagatgttaaatttttgatacataagctttatcatgcaggtgcagtagacgcatttgcgtacgcgttgagtagtaatgcaagaacgcaacaatttcacgctttctacttaagaggacagatcttcgagcagaatatttttctcctagcgcggctttcagaatcttcttaccatctgcaggacatcacatgcaagcacatgaagctggaaccacgcaccattaaatgtaaacctacagacatagctagcacagaaatgctactttggaaacctcggttgcatcggctgacctcggctgttcagcaacgtgagctggttgttgttattgttcatagaatcctgtattttaccagtgtagctttgtccgcccaccccacgcttttggccactagtatgtacgtatatgcgtgggctctatatcattgaaaacgaattttccacgcaaaacccacttcacaccataaaggccttatgacgatgtacattcttcccctcccccctcctcccgaaggaacccttatagtgatcctaatacggtataacgtaaaccggagttgtcagcagagagaagcgaataatatgttttgcgtggggttcccacgagcggcaggaaccgtagcgcattgaaaaatgaatcatcgatcatctttgggtttcaagggttcggacttcgccaaaggtccgaatacttccaccagtatttctctttttccctttccagtctcgttcttcgccgaatccccgctttcccatcatgatcgtttctctgtttctctctttccggaacaggctcttttctgaccgccagttcgtgcggttagttctgtggatcttgcaggcacttgccggtttagccacacttctccgctgtttgtgtcgaatttccctcgagtatgag

SEQ ID NO: 2 - DNA, 345 bp, Saccharomyces cerevisiae
  promoter (1)..(345)
ttatttatgaaaattggcctgtaaatatatacataaataaatatatgcgtcaagctcttttattcggtttgtccagtaatcctcttacaatatatatatatatatatacgcatgtagcgtattcaagaacattacatttaaaaaatagagggtagaacaataaaaatgcacttaacaagttccgtaagtagaaattataatcagtctaagtacaaatttataccttagcttcatttcagttaagggcgatgcaataaaagtggaagaaaaaaaaagaagggaagaggtttgataatatatatatatatatatatataacattgctcagagaaacatacatcagtg

SEQ ID NO: 3 - DNA, 438 bp, Saccharomyces cerevisiae
  promoter (1)..(438)
taaagtgacctggctctatagtgttgtccctctcgcgaggaccattgttgcttgcatatggcttgaaacatatgtcatcacatctgagcgattttacctcttagaattagtttagatatatatgagttgatgaataaatagttataaaaacttgctttggcttcgatatatgaccgttatttttgactaagttttaacgaaggaatctaacctcgttcttgtaattaccaaaatcttcaacaacgcgctgttggaggtatctctatggatgtggcttgaaatatggatgtcttgcctacttctacttctgggaaaggcatttttactcgatcgcgttaatatatgcatcaagaaaataaaaaataaaacgcgaagagctaaaaaaaaaaaagaaaacctactataaataaccgattagaatcgagtttttgtattgaa

SEQ ID NO: 4 - DNA, 209 bp, Saccharomyces cerevisiae
  promoter (1)..(209)
ctttttttttttttttttatagcacgcaactgaaaaaaaaaaaaagaaaaatttttcatcttcgctcgacgtttcttttgtagtactcatctctttttatataaagattaattagttattgtcgctttgcttttccttctttaaaaaatgtttcttgcttttggattttcagatgtcccaagatcattacagtattttaattgaacaaa

SEQ ID NO: 5 - DNA, 361 bp, Saccharomyces cerevisiae
  promoter (1)..(361)
tatcacattccggagtgtcaccccccctctctcaacacagtaatccataaaccagttttacatacacgtaaaaaagaacaggaataaagcttaatcggattattaactcatacgcttgtcacatattgttcgaacaattctggttctttcgagtttcgcagaactttttgaatttttcttttttttctagaacgccgtggaagaaaaacacgcgcatggttttatgagcggttaattctcatcttaataccaaccaggtccttccgccaccccctaaaacatataaatatgcagcttatcccttcaattcttaacatctgtgacctcctcatttcttcccgctgtattagagttcaagaaa

SEQ ID NO: 6 - DNA, 358 bp, Saccharomyces cerevisiae
  promoter (1)..(358)
cacaattgcatacttttccattataactgactgtttcagatcctgcaatagaaagtattttttaagtaatgaaatagtgcttccattgatagtaagtgagtaataagacgatcggaaactattaatataagtatagtttacatatatatatatatatatgtgccagacgataatatcaccgtagtttccggccgggttatgccacgcgaaaacgcctcgagccaagggaaaaactaaactacaaaacaaaacaaaacaaaacaaaaacatctactatatcgttttacccaattcagtatatccgtctacaacagttctcgccacccaagatctgtaaacttacaactgcaaacaaaca

SEQ ID NO: 7 - DNA, 559 bp, Saccharomyces cerevisiae
  promoter (1)..(559)
  misc_feature (4)..(4): n is a, c, g, or t
ggtnacctcacgtcatggaaattttcgccttattcatgttgttgggtatcttcacaaccttgttgatcccagaaactaagagaaagactctagaagaaattaacgagctataccacgatgaaatcgatcctgctacgctaaacttcagaaacaagaataatgacattgaatcttccagcccatctcaacttcaacatgaagcataaaagcctcaaagatgcactaaaacttgtaaactagaacaaataatacaaaaacatttttataaacttattatcaaaccccttacataatctataaatactgtcaggttacatatttattcgataatttcttttaatttcattatttcctcacatctctctgccatcctgttggctggtgccagagcagagcatatcgtcctttcttttttagttccagacgttacccgacatatcatttctcgagcctctggaaaccacgaaacgttttacaaattgcacatctaaaagaaatataaacacagatcaggtatctcataaagtacattaatcgactaagcaagcgacttgagaca

SEQ ID NO: 8 - DNA, 939 bp, Saccharomyces cerevisiae
  promoter (1)..(939)
aagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtccatggtgggtaccgcttatgctcccctccattagtttcgattatataaaaaggccaaatattgtgttattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatg

SEQ ID NO: 9 - DNA, 738 bp, Saccharomyces cerevisiae
  promoter (1)..(738)
caataatccatcatataccattaccctgattcccatcgaagaaaaggcggtgtccccttacccgtccgctcatgccaagagattaattcataaccgctcttccttggatcagaagtgaacatatgaagttgcaactactacatacttactaccgtagtccatcatcaaggacccaaacattcacgactccagcgcgccacgttcttcgccatactgcttaacattttggtacgagtgcgaattagggaagtcgatgataaaatagaatatgcgaaaaagaggaagagcagccgtgagaaaaaagaaaaaaaaaggcctaaggtattctctactccaaaatcgtcgagggagggcaaaagaaattttttttgtttaagggaattgcgaagtcaattgattgatgaagtagcttctctggaaaaatggcggacagacgctgctcaaataaccgtcgaagggggaaaggttatcggtggaaacatatttgtaagtacataatagtgaatttacttcttgtcggcgtcccctaaacgtaattggcggtgtgatggtacttcgttatataaaggtgtgtaatatcctcttttaccatctattatttcttccagcatttcttgctggataacctactgtatccaagctactgggcttttttaaacatacccataacttttttttttttcatttttcgttgctgtgtgctagtacaatttaagcaaaaggaaactgttttgcgtt

SEQ ID NO: 10 - DNA, 736 bp, Aequorea victoria
  CDS (14)..(730)
aagctttattaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcag

SEQ ID NO: 11 - protein, 238 aa, Aequorea victoria
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 12 - DNA, 859 bp, Discosoma sp.
  CDS (54)..(731)
gtttcagccagtgacggtcagtgacagggtgagccacttggtataccaacaaaatgaggtcttccaagaatgttatcaaggagttcatgaggtttaaggttcgcatggaaggaacggtcaatgggcacgagtttgaaatagaaggcgaaggagaggggaggccatacgaaggccacaataccgtaaagcttaaggtaaccaaggggggacctttgccatttgcttgggatattttgtcaccacaatttcagtatggaagcaaggtatatgtcaagcaccctgccgacataccagactataaaaagctgtcatttcctgaaggatttaaatgggaaagggtcatgaactttgaagacggtggcgtcgttactgtaacccaggattccagtttgcaggatggctgtttcatctacaaggtcaagttcattggcgtgaactttccttccgatggacctgttatgcaaaagaagacaatgggctgggaagccagcactgagcgtttgtatcctcgtgatggcgtgttgaaaggagagattcataaggctctgaagctgaaagacggtggtcattacctagttgaattcaaaagtatttacatggcaaagaagcctgtgcagctaccagggtactactatgttgactccaaactggatataacaagccacaacgaagactatacaatcgttgagcagtatgaaagaaccgagggacgccaccatctgttcctttaaggctgaacttggctcagacgtgggtgagcggtaatgaccacaaaaggcagcgaagaaaaaccatgatcgttttttttaggttggcagcctgaaatcgtaggaaatacatcagaaatgttacaaacagg

SEQ ID NO: 13 - protein, 225 aa, Discosoma sp.
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 14 - DNA, 1166 bp, Saccharomyces cerevisiae
  CDS (223)..(1026)
agcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagct

SEQ ID NO: 15 - protein, 267 aa, Saccharomyces cerevisiae
MSKATYKERAATHPSPVAAKLFNIMHEKQTNLCASLDVRTTKELLELVEALGPKICLLKTHVDILTDFSMEGTVKPLKALSAKYNFLLFEDRKFADIGNTVKLQYSAGVYRIAEWADITNAHGVVGPGIVSGLKQAAEEVTKEPRGLLMLAELSCKGSLATGEYTKGTVDIAKSDKDFVIGFIAQRDMGGRDEGYDWLIMTPGVGLDDKGDALGQQYRTVDDVVSTGSDIIIVGRGLFAKGRDAKVEGERYRKAGWEAYLRRCGQQN

SEQ ID NO: 16 - DNA, 4692 bp, Artificial
  sequecne of vector pDsRed1-N1
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 17 - DNA, 45 bp, Artificial
  primer
gagagctagcagactcgagctcttacatacatgtacttataaaac

SEQ ID NO: 18 - DNA, 34 bp, Artificial
  primer
gagaggtaccagttaaagttaatccttctgagag

SEQ ID NO: 19 - DNA, 33 bp, Artificial
  primer
gagagcggccgcctcatactcgagggaaattcg

SEQ ID NO: 20 - DNA, 33 bp, Artificial
  primer
gagaggatccggtaatctgcgtcttgccatcag

SEQ ID NO: 21 - DNA, 12 bp, Artificial
  primer
cggctggttcta

SEQ ID NO: 22 - DNA, 22 bp, Artificial
  primer
gtcgactacgtcgttaaggccg

SEQ ID NO: 23 - DNA, 23 bp, Artificial
  primer
acaaagctcctctcctgctcaag

SEQ ID NO: 24 - DNA, 22 bp, Artificial
  primer
actaggatgagtagcagcacgt